SEQUENCE LISTING (2 sequences)

SEQ ID NO: 1 - DNA, 1377 bp, Artificial Sequence
  Synthetic
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 458 aa, Artificial Sequence
  Synthetic
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0138032 A1
(43) Pub. Date: May 13, 2021

(54) NON-ANTIBODY VEGF ANTAGONISTS FOR THE TREATMENT OF NEOVASCULAR GLAUCOMA

(71) Applicant: BAYER HEALTHCARE LLC, Whippany, NJ (US)

(72) Inventors: Sergio Casimiro SILVA LEAL, Reinach (CH); Oliver ZEITZ, Berlin (DE); Yuji IWAMOTO, Osaka (JP); Masato KOBAYASHI, Tokyo (JP)

(73) Assignee: BAYER HEALTHCARE LLC, Whippany (US)

(21) Appl. No.: 16/622,633

(22) PCT Filed: Jun. 12, 2018
(86) PCT No.: PCT/EP2018/065464

§ 371 Date: Dec. 13, 2019

(30) Foreign Application Priority Data
Jun. 14, 2017 (EP) 17176072.1

(51) Int. Cl. A61K 38/17; A61K 45/06; A61K 9/00; A61P 27/06

(52) U.S. Cl. CPC A61K 38/179; A61P 27/06; A61K 9/0019; A61K 45/06

(57) ABSTRACT

The present invention relates to methods of treating the manifestation of NVG including increased intraocular pressure and anterior segment neovascularization with a non-antibody VEGF antagonist.

The present invention relates to methods of treating the manifestations of Neovascular glaucoma (NVG) including increased intraocular pressure and anterior segment neovascularization with a non-antibody VEGF antagonist.

### BACKGROUND

Neovascular glaucoma (NVG) is a severe form of glaucoma attributed to new blood vessels obstructing aqueous humor outflow, secondary to ocular ischemia. Clinical conditions associated with ischemia such as proliferative diabetic retinopathy, ischemic central retinal vein occlusion, and ocular ischemic syndrome are the most common entities associated with the development of NVG.

The ocular ischemia triggers the production of pro-angiogenic factors in the retina which eventually diffuse into the anterior chamber and lead the development of neovascularization (NV) in the anterior chamber angle (NVA) and the iris (NVI). As a result, a fibrovascular membrane forms in the iris, the anterior chamber angle, or both. The development of this membrane obstructs the aqueous humor outflow and causes a significant intraocular pressure (IOP) elevation, which is difficult to control with conventional IOP lowering therapies. Panretinal photocoagulation (PRP) is still the gold standard therapy for those cases in whom NVG arises from an ischemic retina. PRP destroys the ischemic tissue responsible for the vasoproliferative stimulus, reducing the global oxygen demand of the retina as well as eliminating the synthesis of vasoproliferative factors. However, PRP damages healthy tissues that are not involved in the process of hypoxia-induced neovascularization. Therefore, there is a need to develop specific targeted therapies that will reduce angiogenic factors and subsequent neovascularization while at the same time preserving healthy retinal cells. Early evidence shows that inhibition of VEGF is promising in that respect (for review see Guerrero et al. 2017). Several therapies have been developed with the aim of inhibiting VEGF and optimizing the management of several ocular pathologies. These therapeutic applications include VEGF inhibitors such as:

| | |
|---|---|
| Aflibercept (Eylea ®) | WO2000/75319 |
| Bevacizumab (Avastin ®) | WO 9845331 |
| Ranibizumab (Lucentis ®) | WO9845331 |
| Pegaptanib (Macugen ®) | WO9818480 |
| KH-902/conbercept (Langmu ®) | WO2005121176 |

The efficacy of bevacizumab, ranibizumab and aflibercept in the treatment of NVG was investigated in clinical studies. 26 patients with NVG received 3 intravitreal injections of 2.5 mg (0.1 mL) bevacizumab at monthly intervals. At 1, 3, and 6 months after intervention NV in the iris was reduced and IOP was decreased (Yazdani et al. 2009).

In another study patients with NVG (n=10) were injected intravitreally at baseline with 0.5 mg ranibizumab, then—if necessary—on a monthly basis. A significantly improved IOP was evident at the first follow-up visit in 8 patients. After month 7, the IOP of all patients examined was in normal range and maintained up to 12 month. Fourteen days after initial injection, seven patients of the NVG group presented with a complete regression of rubeosis. After 12 month a partial reduction of rubeosis was observed in four patients and a complete reduction in the remaining cases (n=6) at the last follow-up (Lueke et al. 2013).

SooHoo et al. (2015) reported on 4 patients with newly diagnosed stage 1 NVG (Rubeosis iridis) or stage 2 NVG (open angle glaucoma). Patients with stage 3 NVG angle glaucoma were not included into the study. The patients were treated with intravitreal aflibercept at the time of diagnosis, at 4 weeks, 8 weeks and then every 8 weeks thereafter up until 52 weeks. Regression of NV of the iris and angle was observed by 1 week after injection and no recurrence of NV could be detected up to week 52. IOP decreased or stabilized by 1 week after injection and was maintained up to week 52.

WO2014 033184 (Novartis) relates to the use of non-antibody anti-VEGF-agents in the treatment of eye diseases. Among others the use of non-antibody anti-VEGF-agents in the treatment of NVG is described.

However, there is still a need to treat patients with NVG especially of patients with peripheral anterior synechiae and/or closure of the anterior chamber angle with reduced number of intravitreal injections, in order to reduce treatment related patient burden and to reduce adverse events and complications associated with intravitreal injections.

### SUMMARY OF INVENTION

It has now been found, that a single intravitreal injection of a non-antibody VEGF antagonist, such as aflibercept, surprisingly reduces the IOP and decreases the anterior segment neovascularization, such as the neovascularization of the iris (NVI) and anterior chamber angle (NVA), in patients with all stages of NVG over a period of 13 weeks.

The present invention provides non-antibody VEGF antagonists for use in the treatment of the manifestation of NVG including increased intraocular pressure and anterior segment neovascularization.

The present invention further provides the use of non-antibody VEGF antagonists in a method of treatment of the manifestation of NVG including increased intraocular pressure and anterior segment neovascularization.

The present invention provides the use of non-antibody VEGF antagonists for the preparation of a pharmaceutical composition, preferably a medicament, for the treatment of the manifestation of NVG including increased intraocular pressure and anterior segment neovascularization.

### DETAILED DESCRIPTION OF THE INVENTION

### Definition of Terms

The expression “neovascular glaucoma” (NVG) as used herein, means elevated intraocular pressure and/or optic nerve damage which results from elevation in intraocular pressure, caused by growth of new vessels which affect structures involved in regulating the flow of aqueous humor in the eye. Synonyms of NVG are hemorrhagic glaucoma, congestive glaucoma, thrombotic glaucoma, and rubeotic glaucoma. Some specific forms of secondary glaucoma are also synonyms with neovascular glaucoma, specifically secondary glaucoma due to proliferative diabetic retinopathy, retinal vein occlusions and ocular ischemic syndrome.

Several classifications have been proposed for the staging of NVG.

Weiss and Gold (1978) proposed a classification of anterior segment neofibrovascularization which has been used in several studies:

TABLE 1
Grading systems for neovascularization of the iris (NVI) and anterior chamber angle (NVA)

| Grade | Neovascularization of Iris | Neovascularization of Angle |
|---|---|---|
| 0 | No iris neovascularization | No angle neovascularization |
| 1 | Fine surface neovascularization of the | Fine neovascular twigs crossing the scleral |
| | pupillary zone of the iris involving less than | spur and ramifying on the trabecular |
| | two quadrants. | meshwork involving less than two quadrants. |
| 2 | Surface neovascularization of the pupillary | Neovascular twigs crossing the scleral spur |
| | zone of the iris involving more than two | and ramifying on the trabecular meshwork |
| | quadrants. | involving more than two quadrants. |
| 3 | In addition to neovascularization of the | In addition to neovascularization of the |
| | pupillary zone, neovascularization of the | trabecular meshwork, peripheral anterior |
| | ciliary of the iris and/or ectropion uveae | synechiae (PAS) involving one to three |
| | involving one to three quadrants. | quadrants. |
| 4 | In addition to neovascularization of the | In addition to neovascularization of the |
| | pupillary zone, neovascularization of the | trabecular meshwork, PAS involving more |
| | ciliary zone of the iris and/or ectropion uveae | than three quadrants. |
| | involving more than three quadrants. | |

Another way of classification is a staging of NVG:

Stage 1: Rubeosis iridis—isolated neovascularization of the iris without IOP elevation

Stage 2: Open angle glaucoma—anterior segment neovascularization and elevation of IOP

Stage 3: Closed angle glaucoma—peripheral anterior synechiae and/or closure of the anterior chamber angle together with elevation of IOP

The expression “anterior segment neovascularization” or “anterior segment neofibrovascularization” as used herein means growth of new vessels in the anterior segment of the eye, which constitutes the space extending from the cornea anteriorly to the lens posteriorly, and contains the anterior chamber angle, iris, pupil, ciliary body and ciliary processes and aqueous humor, among other structures. It includes, but is not limited to, the neovascularization of the anterior chamber angle (NVA) and the neovascularization of the iris (NVI).

The expression “intraocular pressure” (IOP) as used herein means elevation of the pressure of the aqueous humor inside the eye. Since the direct measurement of intraocular pressure requires perforation of the eye, in clinical practice the intraocular pressure is measured indirectly through the cornea using a variety of strategies such as applanation, indentation and rebound or others.

The term “treating” or “treatment” as used in the present text is used conventionally, e.g. the management or care of a subject for the purpose of combating, alleviating, reducing, relieving, improving the condition of NVG. The term “therapeutic” as used in the present text means that the non-antibody VEGF antagonist binds to a VEGF-ligand or VEGF receptor, and produces a change in the symptoms or conditions associated with NVG, including IOP, NVA, and NVI. It is sufficient that a therapeutic dose produces an incremental change in the symptoms or conditions associated with the disease; a cure or complete remission of symptoms is not required.

The phrase “immediately preceding dose” as used herein, means, in a sequence of multiple administrations, the administration of non-antibody VEGF antagonist to a patient prior to the administration of the very next dose in the sequence with no intervening doses.

The term “VEGF” as used herein refers to vascular endothelial growth factor family comprising five members VEGF-A, placenta growth factor (PGF), VEGF-B, VEGF-C and VEGF-D.

As used herein, the expression “VEGF antagonist” means any molecule that blocks, reduces, neutralizes, inhibits, abrogates, or interferes with the normal biological activity of VEGF including its binding to one or more VEGF receptors (VEGFR1 and VEGFR2). VEGF antagonists include for example molecules which interfere with the interaction between VEGF and a natural VEGF receptor, e.g. molecules which bind to VEGF or a VEGF receptor and prevent or otherwise hinder the interaction between VEGF and a VEGF receptor. VEGF antagonists include

(i) antibody VEGF antagonists such as but not limited to
anti-VEGF antibodies such as bevacizumab (Avastin®; WO 9845331) and antigen-binding fragments thereof such as ranibizumab (Lucentis® WO9845331),
anti-VEGFR1 or anti-VEGFR2 antibodies or and antigen-binding fragments thereof

(ii) non-antibody VEGF antagonist such as but not limited to
small molecule inhibitors of the VEGFR tyrosine kinases (e.g. sunitinib),
RNA aptamers specific to VEGF,
Antibody-Mimetika against VEGF or VEGF receptors (e.g. Affibody®-molecules (e.g. DARPin® MP0112 (WO2010/060748)), Affiline, Affitine, Anticaline, Avimere), and
VEGF receptor-based chimeric molecules also known as VEGF fusion proteins or VEGF-Traps such as aflibercept (Eylea®; WO2000/75319) or conbercept (Langmu®, WO2005121176).
   Treatment of Patients Diagnosed with NVG

Non-antibody VEGF antagonist such as aflibercept have surprisingly been found to reduce the IOP and to decrease the anterior segment neovascularization such as the NVA and NVI in patients with all stages of NVG over a period of 13 weeks after a single intravitreal injection.

In accordance with a first aspect, the present invention covers non-antibody VEGF antagonists for use in the treatment of the manifestation of NVG including increased intraocular pressure and anterior segment neovascularization.

In accordance with a further aspect, the present invention covers the use of non-antibody VEGF antagonists for the treatment of the manifestation of NVG including increased intraocular pressure and anterior segment neovascularization.

In accordance with a further aspect, the present invention covers the use of non-antibody VEGF antagonists in a method of treatment of the manifestation of NVG including increased intraocular pressure and anterior segment neovascularization.

In accordance with a further aspect, the present invention covers use of non-antibody VEGF antagonists for the preparation of a pharmaceutical composition, preferably a medicament, for the treatment of the manifestation of NVG including increased intraocular pressure and anterior segment neovascularization.

In accordance with a further aspect, the present invention covers a method of treatment of the manifestation of NVG including increased intraocular pressure and anterior segment neovascularization, using an effective amount of non-antibody VEGF antagonists.

### Patients

According to the invention the patients are diagnosed with NVG. This includes the measurement of the IOP, which is the fluid pressure inside the eye, by the use of tonometry and the assessment of the eye to detect presence of neovascularization in the iris and/or the anterior chamber angle. The assessment of the eye may be performed by examination by the healthcare practitioner, including gonioscopy for observation of the anterior chamber angle, or by specialized exams such as fluorescein angiography.

According to the invention, patients of all stages of NVG can be treated.

In accordance with another embodiment of all aspects, the present invention covers non-antibody VEGF antagonists for use in the treatment of the manifestation of NVG including increased intraocular pressure and anterior segment neovascularization wherein the anterior segment neovascularization is of NVI of grade 3 or 4 or/and NVA of grade 3 or 4.

In accordance with another embodiment of all aspects, the present invention covers non-antibody VEGF antagonists for use in the treatment of the manifestation of NVG including increased intraocular pressure and anterior segment neovascularization wherein the treatment is administered to a subject who has been established to have NVI of grade 3 or 4 or/and NVA of grade 3 or 4.

In accordance with another embodiment of all aspects, the present invention covers non-antibody VEGF antagonists for use in the treatment of the manifestation of NVG including increased intraocular pressure and anterior segment neovascularization wherein the treatment is administered to a subject who has been established to have peripheral anterior synechiae and/or closure of the anterior chamber angle.

In accordance with another embodiment of all aspects, the present invention covers non-antibody VEGF antagonists for use in the treatment of the manifestation of NVG including increased intraocular pressure and anterior segment neovascularization wherein the treatment is administered to a subject who has been established to have stage 3 NVG.

According to the invention the patients can be treatment naïve or be pre-treated for example with laser photocoagulation, systemic or topical IOP lowering drugs, glaucoma laser or laser trabeculoplasty.

### Treatment Regimens

In some cases, a single injection of the non-antibody VEGF antagonist may be sufficient to stabilize the IOP to a value below 21 mmHg and to achieve absence anterior segment neovascularization.

In accordance with another embodiment of all aspects, the present invention covers non-antibody VEGF antagonists for use in the treatment of the manifestation of NVG including increased intraocular pressure and anterior segment neovascularization wherein said method comprises,
i.) a single initial dose of the non-antibody VEGF antagonist and
ii) one or more secondary doses which are administered 5, 6, 7, 8, or 9 weeks after the immediately preceding dose to the subject who has been established to have an IOP of higher than 21 mmHg and a persistent or incomplete regression of anterior segment neovascularization at 5, 6, 7, 8, or 9 weeks after the immediately preceding dose.

In other cases, more than a single injection each one 5, 6, 7, 8, or 9 weeks, preferably 5, 8, or 9 week apart are administered to the patient. In certain cases, two injections spaced 5, 6, 7, 8, or 9 weeks apart, preferably 5, 8, or 9 weeks apart may be required to improve or halt disease progression. Treatment may be continued until normal IOP below 21 mmHg and absence anterior segment neovascularization is achieved.

In accordance with another embodiment of all aspects, the present invention covers non-antibody VEGF antagonists for use in the treatment of the manifestation of NVG including increased intraocular pressure and anterior segment neovascularization wherein one secondary dose is administered 5, 8, or 9 weeks after the single initial dose to the subject who has been established to have an IOP of higher than 21 mmHg and a persistent or incomplete regression of anterior segment neovascularization at 5, 8, or 9 weeks after the single initial dose.

In accordance with another embodiment of all aspects, the present invention covers non-antibody VEGF antagonists for use in the treatment of the manifestation of NVG including increased intraocular pressure and anterior segment neovascularization wherein said treatment is combined with a IOP lowering therapy.

Combining non-antibody VEGF antagonist therapy with therapies commonly used for treatment of NVG may reduce the total treatment time as well as increase the patient benefit. According to the invention, said therapies comprise one or more systemic or topical therapies and are administered in accordance to the instructions in the label of the respective medication.

Examples for systemic IOP-lowering therapy are:
Carbonic anhydrase inhibitors
Intravenous hyperosmotic agents

Examples for topical IOP-lowering drugs are from the following classes:
Prostaglandin (PG) analog
Sympatholytic agent
Carbonic anhydrase inhibitor (CAI)
Sympathomimetic agent
Rho-kinase inhibitor

Examples for other interventions are the following:
Laser Panretinal Photocoagulation
Laser Iridotomy
Laser Trabeculoplasty
Surgical procedures aimed at controlling increased intraocular pressure, such as trabeculectomy or implantation of devices such as valves or shunts.

In accordance with another embodiment of all aspects, the present invention covers non-antibody VEGF antagonists for use in the treatment of the manifestation of NVG including increased intraocular pressure and anterior segment neovascularization wherein said IOP lowering therapy is selected from the group of
Carbonic anhydrase inhibitors
Intravenous hyperosmotic agents
Prostaglandin (PG) analog
Sympatholytic agent
Carbonic anhydrase inhibitor (CAI)
Sympathomimetic agent
Rho-kinase inhibitor
Laser Panretinal Photocoagulation
Laser Iridotomy
Laser Trabeculoplasty
Surgical procedures aimed at controlling increased intraocular pressure, such as trabeculectomy or implantation of devices such as valves or shunts.

### Non-Antibody VEGF Antagonists

The present invention comprises administering to a patient a non-antibody VEGF antagonist for the treatment of NVG. Non-antibody VEGF antagonists include but are not limited to
small molecule inhibitors of the VEGFR tyrosine kinases (e.g. sunitinib),
RNA aptamers specific to VEGF,
Antibody-Mimetika against VEGF or VEGF receptors (e.g. Affibody®-molecules (e.g. DARPin® MP0112 (WO2010/060748)), Affiline, Affitine, Anticaline, Avimere), and
VEGF receptor-based chimeric molecules also known as VEGF fusion proteins or VEGF-Traps such as aflibercept (Eylea®; WO2000/75319) or conbercept (Langmu®, WO2005121176).

VEGF receptor-based chimeric molecules include chimeric polypeptides which comprise two or more immunoglobulin (Ig)-like domains of a VEGF receptor such as VEGFR1 (also referred to as Flt 1) and/or VEGFR2 (also referred to as Flk1 or KDR), and may also contain a multimerizing domain, e.g. a Fc domain which facilitates the multimerization, e.g. dimerization of two or more chimeric polypeptides. Exemplary VEGF receptor-based chimeric molecules are aflibercept or conbercept.

Aflibercept (WO2000/75319; Regeneron) is a recombinant protein created by fusing the second Ig domain of human VEGFR1 with the third Ig domain of human VEGFR2, which is in turn fused to the constant region of human IgG1. It is encoded by the nucleic acid sequence of SEQ ID NO:1 and comprises three components: (1) a VEGFR1 component comprising amino acids 27 to 129 of SEQ ID NO:2; (2) a VEGFR2 component comprising amino acids 130 to 231 of SEQ ID NO:2; and (3) a multimerization component comprising amino acids 232 to 457 of SEQ ID NO:2 (the C-terminal amino acid of SEQ ID NO:2 [i.e., K458] may or may not be included in the VEGF antagonist used in the methods of the invention; see e.g. U.S. Pat. No. 7,396,664). Amino acids 1-26 of SEQ ID NO:2 are the signal sequence. Additional VEGF receptor based chimeric molecules which can be used in the context of the present invention are disclosed in U.S. Pat. Nos. 7,396,664, 7,303,746 and WO 00/75319.

nucleic acid sequence
SEQ ID NO: 1
ATGGTCAGCTACTGGGACACCGGGGTCCTGCTGTGCGCGCTGCT
CAGCTGTCTGCTTCTCACAGGATCTAGTTCCGGAAGTGATACCG
GTAGACCTTTCGTAGAGATGTACAGTGAAATCCCCGAAATTATA
CACATGACTGAAGGAAGGGAGCTCGTCATTCCCTGCCGGGTTAC
GTCACCTAACATCACTGTTACTTTAAAAAAGTTTCCACTTGACA
CTTTGATCCCTGATGGAAAACGCATAATCTGGGACAGTAGAAAG
GGCTTCATCATATCAAATGCAACGTACAAAGAAATAGGGCTTCT
GACCTGTGAAGCAACAGTCAATGGGCATTTGTATAAGACAAACT
ATCTCACACATCGACAAACCAATACAATCATAGATGTGGTTCTG
AGTCCGTCTCATGGAATTGAACTATCTGTTGGAGAAAAGCTTGT
CTTAAATTGTACAGCAAGAACTGAACTAAATGTGGGGATTGACT
TCAACTGGGAATACCCTTCTTCGAAGCATCAGCATAAGAAACTT
GTAAACCGAGACCTAAAAACCCAGTCTGGGAGTGAGATGAAGAA
ATTTTTGAGCACCTTAACTATAGATGGTGTAACCCGGAGTGACC
AAGGATTGTACACCTGTGCAGCATCCAGTGGGCTGATGACCAAG
AAGAACAGCACATTTGTCAGGGTCCATGAAAAGGACAAAACTCA
CACATGCCCACCGTGCCCAGCACCTGAACTCCTGGGGGGACCGT
CAGTCTTCCTCTTCCCCCCAAAACCCAAGGACACCCTCATGATC
TCCCGGACCCCTGAGGTCACATGCGTGGTGGTGGACGTGAGCCA
CGAAGACCCTGAGGTCAAGTTCAACTGGTACGTGGACGGCGTGG
AGGTGCATAATGCCAAGACAAAGCCGCGGGAGGAGCAGTACAAC
AGCACGTACCGTGTGGTCAGCGTCCTCACCGTCCTGCACCAGGA
CTGGCTGAATGGCAAGGAGTACAAGTGCAAGGTCTCCAACAAAG
CCCTCCCAGCCCCCATCGAGAAAACCATCTCCAAAGCCAAAGGG
CAGCCCCGAGAACCACAGGTGTACACCCTGCCCCCATCCCGGGA
TGAGCTGACCAAGAACCAGGTCAGCCTGACCTGCCTGGTCAAAG
GCTTCTATCCCAGCGACATCGCCGTGGAGTGGGAGAGCAATGGG
CAGCCGGAGAACAACTACAAGACCACGCCTCCCGTGCTGGACTC
CGACGGCTCCTTCTTCCTCTACAGCAAGCTCACCGTGGACAAGA
GCAGGTGGCAGCAGGGGAACGTCTTCTCATGCTCCGTGATGCAT
GAGGCTCTGCACAACCACTACACGCAGAAGAGCCTCTCCCTGTC
TCCGGGTAAATGA
amino acid sequence:
SEQ ID NO: 2
MVSYWDTGVLLCALLSCLLLTGSSSGSDTGRPFVEMYSEIPEII
HMTEGRELVIPCRVTSPNITVTLKKFPLDTLIPDGKRIIWDSRK
GFIISNATYKEIGLLTCEATVNGHLYKTNYLTHRQTNTIIDVVL
SPSHGIELSVGEKLVLNCTARTELNVGIDFNWEYPSSKHQHKKL
VNRDLKTQSGSEMKKFLSTLTIDGVTRSDQGLYTCAASSGLMTK
KNSTFVRVHEKDKTHTCPPCPAPELLGGPSVFLFPPKPKDTLMI
SRTPEVTCVVVDVSHEDPEVKFNWYVDGVEVHNAKTKPREEQYN
STYRVVSVLTVLHQDWLNGKEYKCKVSNKALPAPIEKTISKAKG
QPREPQVYTLPPSRDELTKNQVSLTCLVKGFYPSDIAVEWESNG
QPENNYKTTPPVLDSDGSFFLYSKLTVDKSRWQQGNVFSCSVMH
EALHNHYTQKSLSLSPGK

In accordance with another embodiment of all aspects, the present invention covers non-antibody VEGF antagonists for use in the treatment of the manifestation of NVG including increased intraocular pressure and anterior segment neovascularization wherein said non-antibody VEGF antagonists comprise a VEGF fusion protein or preferably aflibercept.

In accordance with another embodiment of all aspects, the present invention covers non-antibody VEGF antagonists for use in the treatment of the manifestation of NVG including increased intraocular pressure and anterior segment neovascularization wherein said non-antibody VEGF antagonists comprise a VEGF fusion protein encoded by the nucleic acid sequence of SEQ ID NO: 1.

In accordance with another embodiment of all aspects, the present invention covers a non-antibody VEGF antagonists for use in the treatment of the manifestation of NVG including increased intraocular pressure and anterior segment neovascularization wherein said non-antibody VEGF antagonists comprise a VEGF fusion protein comprising (1) a VEGFR1 component comprising amino acids 27 to 129 of SEQ ID NO:2; (2) a VEGFR2 component comprising amino acids 130 to 231 of SEQ ID NO:2; and (3) a multimerization component comprising amino acids 232 to 457 of SEQ ID NO:2.

### Pharmaceutical Formulation of Non-Antibody VEGF Antagonist

The present invention includes methods in which the non-antibody VEGF antagonist that is administered to the patient is contained within a pharmaceutical formulation. Non-antibody VEGF antagonist of the invention will generally be administered to the patient as liquid solution, though other formulations may be used, such as a slow-release depot or eye drops.

The pharmaceutical formulation may comprise the non-antibody VEGF antagonist along with at least one inactive pharmaceutically suitable excipients. Pharmaceutically suitable excipients include, inter alia,
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),

Any of the foregoing mixtures may be appropriate in the context of the methods of the present invention, provided that the non-antibody VEGF antagonist is not inactivated by the formulation and the formulation is physiologically compatible and tolerable with the route of administration.

Pharmaceutical formulations useful for administration by injection in the context of the present invention may be prepared by dissolving, suspending or emulsifying a non-antibody VEGF antagonist in a sterile aqueous medium, for example, physiological saline, an isotonic solution containing glucose or sucrose and other auxiliary agents, etc., which may be used in combination with an appropriate solubilizing agent such as an alcohol (e.g. ethanol), a polyalcohol (e.g. propylene glycol, polyethylene glycol), a nonionic surfactant [e.g. polysorbate 80, HCO-50 (polyoxyethylene (50 mol) adduct of hydrogenated castor oil)] etc. The injection thus prepared can be filled in an appropriate ampoule or syringe if desired.

For example, aflibercept is generally administered via intravitreal injection at a dose of 2 mg suspended in 0.05 mL buffer comprising 40 mg/mL in 10 mM sodium phosphate, 40 mM sodium chloride, 0.03% polysorbate 20, and 5% sucrose, pH 6.2.

### Modes of Administration

The non-antibody VEGF antagonist or pharmaceutical formulation comprising the non-antibody VEGF antagonist may be administered to the patient by any known delivery system and/or administration method. In certain embodiments, the non-antibody VEGF antagonist is administered to the patient by ocular or intraocular administration. Intraocular administration includes, for example, intravitreal, subretinal, subscleral, intrachoroidal, subconjunctival, retrobulbar, and subtenon. Suitable intraocular administration forms are those according to the prior art which function by releasing the active compound rapidly and/or in a modified or controlled manner and which contain the active compound in a crystalline and/or amorphous and/or dissolved form, such as for example, injections and concentrates for injections (including, for example, solutions, suspensions, vesicular/colloidal systems, emulsions), powder for injections (including, for example, milled compound, blends, lyophilisates, precipitates), gels for injections (semi-solid preparations including, for example, hydrogels, in-situ-forming hydrogels) and implants (solid preparations including, for example, biodegradable and non-degradable implants, implantable pumps).

In other embodiments the non-antibody VEGF antagonist can be administered to the patient by topical administration, e.g., via eye drops or other liquid, gel, slow-release depot, ointment or fluid which contains the non-antibody VEGF antagonist and can be applied directly to the eye.

### REFERENCES

Guerrero et al. Current Perspectives on the Use of Anti-VEGF Drugs as Adjuvant Therapy in Glaucoma. Adv Ther (2017) 34:378-395
SooHoo J R, Seibold L K, Pantcheva M B, Kahook M Y. Aflibercept for the treatment of neovascular glaucoma. Clin Experiment Ophthalmol. 2015; 43(9):803-7
Lueke J, Nassar K, Lueke M, Grisanti S. Ranibizumab as adjuvant in the treatment of rubeosis iridis and neovascular glaucoma—results from a prospective interventional case series. Graefes Arch Clin Exp Ophthalmol. 2013; 251(10):2403-13.
Yazdani S, Hendi K, Pakravan M, Mandavi M, Yaseri M. Intravitreal bevacizumab for neovascular glaucoma: a randomized controlled trial. J Glaucoma. 2009; 18(8):632-7.
Weiss D I and Gold D. Neofibrovascularization of Iris and Anterior Chamber Angle: A Clinical Classification Annals of Ophthalmology 1978; 10(1):488-491.

### Example 1

### Study Design

The efficacy of aflibercept in comparison to sham treatment was studied in randomized, double-masked, and controlled study with 54 subjects diagnosed with NVG with neovascularization in the anterior segment of both iris and anterior chamber angle and with IOP higher than 25 mmHg in the study eye due to anterior segment (both iris and anterior chamber angle) neovascularization. 8 of the 54 subjects were diagnosed with stage 3 NVG having grade 4 NVA with PAS involving more than 3 quadrants.
Aflibercept group: Subjects were administered with 2 mg (0.05 mL) aflibercept on Day 1. They could receive sham injection at Week 1, followed by PRN administration of aflibercept at week 5 and 9 according to the retreatment criteria (2 mg (0.05 mL) aflibercept injection at Week 5 and/or Week 9 when all the re-treatment criteria were met).
Sham group: Subjects were administered with a sham injection on Day 1. Subsequently, subjects received a single injection of 2 mg of Eylea at Week 1 followed by PRN administration at Weeks 5 and 9 according to the retreatment criteria (2 mg (0.05 mL) aflibercept injection at Week 1, Week 5 and/or Week 9 when all the re-treatment criteria were met).

Re-Treatment Criteria:
IOP higher than 21 mmHg,
Incomplete regression of iris neovascularization
Aflibercept treatment deemed necessary by the investigator
Background Treatment: All subjects were additionally treated with standard therapy including IOP-lowering drug and panretinal photocoagulation given concomitantly with test drug.

### Result.

### Change in IOP:

The difference between the treatment groups in least square mean change of IOP from baseline to Week 1 was 4.9 mmHg, with a 95% CI of 10.2 to 0 3 mmHg with an upper limit of the CI above zero (p=0.0644, analysis of covariance model, including treatment group and stage of NVG for randomization as fixed effect and baseline IOP as a covariate). Thus, the superiority of the aflibercept group over the sham group was not demonstrated statistically. However, the change in IOP in the aflibercept group was −9.9 mmHg (LS mean change), which was comparable to the expected clinically meaningful reduction used to design the study (assumption for the determination of sample size: mean±SD of 10±12 mmHg for the aflibercept group).

Among the planned sensitivity analyses, PPS analysis provided the upper limit of the 95% CI lower than zero (LS mean difference in change in IOP was −5.5 mmHg with 95% CI of −10.8 to −0.2, p=0.0423), showing clinical significance.

This shows that the proportion of subjects in whom IOP could be controlled was much higher in the aflibercept group than in the sham group at Week 1.

The proportion of subjects in whom the IOP was controlled (≤21 mmHg) in the aflibercept group was 44.4% at Week 1 and increased up to 76.9% at Week 9. The proportion was then maintained until Week 13 (73.1%). In the sham group the proportion of subjects in whom the IOP was controlled was only 7.4% at Week 1. However, subsequent to the first administration of aflibercept at Week 1, it increased to 63.0% at Week 2. Also in the sham group the proportion increased up to 85.2% at Week 9 and was then maintained until Week 13 (84.6%).

### Change in NVI:

The proportion of subjects with improvement in NVI grade at Week 1 was 70.4% in the aflibercept group and 11.5% in the sham group. The point estimate of MH-adjusted difference was 59.1% with a 95% CI of 37.0% to 81.2%. The NVI grade was stable in 29.6% of subjects and worsened in no subject in the aflibercept group, while stable in 80.8% and worsened in 7.7% in the sham group.

This shows that the proportion of subjects who had improved NVI grade from baseline to Week 1 was markedly greater in the aflibercept group than in the sham group.

After Week 1, the NVI grade was further improved until Week 13 in the aflibercept group. In the sham group, subsequent to the first administration of aflibercept at Week 1, the NVI grade was improved in most of the subjects (69.2%) at Week 2. The NVI grade was improved until Week 13 in the sham group as well.

### Change in NVA:

The proportion of subjects with improvement in NVA grade at Week 1 was 59.3% in the aflibercept group and 11.5% in the sham group. The point estimate (two-sided 95% CI) of MH-adjusted difference was 48.3% with a 95% CI of 26.4% to 70.1%. The NVA grade was stable in 40.7% of subjects and worsened in no subject in the aflibercept group, while stable in 76.9% and worsened in 11.5% in the sham group.

This shows that the proportion of subjects who had improved NVA grade from baseline to Week 1 was markedly greater in the aflibercept group than in the sham group.

After Week 1, in the aflibercept group, the proportion of subject with an improved NVA grade further increased up to 80.8% at Week 9 and was then maintained until Week 13. In the sham group, subsequent to the first administration of aflibercept at Week 1, the NVA grade was improved in most of the subjects (53.8%) at Week 2. Also in the sham group the proportion of subject with an improved NVA grade further increased up to 81.5% at Week 9 and was then maintained until Week 13.

1) A non-antibody VEGF antagonist for use in the treatment of the manifestation of NVG including increased intraocular pressure and anterior segment neovascularization.

2) A non-antibody VEGF antagonist for use in the treatment of the manifestation of NVG including increased intraocular pressure and anterior segment neovascularization
according to claim 1
wherein the treatment is administered to a subject who has been established to have neovascularization of the iris (NVI) of grade 3 or 4 or/and anterior chamber angle (NVA) of grade 3 or 4.

3) A non-antibody VEGF antagonist for use in the treatment of the manifestation of NVG including increased intraocular pressure and anterior segment neovascularization
according to claim 1
wherein the treatment is administered to a subject who has been established to have peripheral anterior synechiae and/or closure of the anterior chamber angle.

4) A non-antibody VEGF antagonist for use in the treatment of the manifestation of NVG including increased intraocular pressure and anterior segment neovascularization
according to claim 1
wherein said method comprises sequentially administering to the subject
i.) a single initial dose of the non-antibody VEGF antagonist
ii) one or more secondary doses which are administered 5, 6, 7, 8, or 9 weeks after the immediately preceding dose to the subject who has been established to have an IOP of higher than 21 mmHg and a persistent or incomplete regression of anterior segment neovascularization at 5, 6, 7, 8, or 9 weeks after the immediately preceding dose.

5) A non-antibody VEGF antagonist for use in the treatment of the manifestation of NVG including increased intraocular pressure and anterior segment neovascularization according to claim 4
wherein one secondary dose is administered 5, 8, or 9 weeks after the single initial dose to the subject who has been established to have an TOP of higher than 21 mmHg and a persistent or incomplete regression of anterior segment neovascularization at 5, 8, or 9 weeks after the single initial dose.

6) A non-antibody VEGF antagonist for use in the treatment of the manifestation of NVG including increased intraocular pressure and anterior segment neovascularization
according to claim 1
wherein said treatment is combined with TOP lowering therapy.

7) A non-antibody VEGF antagonist for use in the treatment of the manifestation of NVG including increased intraocular pressure and anterior segment neovascularization according to claim 6
wherein said TOP lowering therapy is selected from the group of
Carbonic anhydrase inhibitors
Intravenous hyperosmotic agents
Prostaglandin (PG) analog
Sympatholytic agent
Carbonic anhydrase inhibitor (CAI)
Sympathomimetic agent
Rho-kinase inhibitor
Laser Panretinal Photocoagulation
Laser Iridotomy
Laser Trabeculoplasty
Surgical procedures aimed at controlling increased intraocular pressure, such as trabeculectomy or implantation of devices such as valves or shunts.

8) A non-antibody VEGF antagonist for use in the treatment of the manifestation of NVG including increased intraocular pressure and anterior segment neovascularization
according to claim 1,
wherein said non-antibody VEGF antagonist comprises a VEGF fusion protein or preferably aflibercept.

9) A non-antibody VEGF antagonist for use in the treatment of the manifestation of NVG including increased intraocular pressure and anterior segment neovascularization
according to claim 1
wherein said non-antibody VEGF antagonist comprises a VEGF fusion protein encoded by the nucleic acid sequence of SEQ ID NO: 1

10) A non-antibody VEGF antagonist for use in the treatment of the manifestation of NVG including increased intraocular pressure and anterior segment neovascularization
according to claim 1
wherein said non-antibody VEGF antagonist comprises a VEGF fusion protein comprising (1) a VEGFR1 component comprising amino acids 27 to 129 of SEQ ID NO:2; (2) a VEGFR2 component comprising amino acids 130 to 231 of SEQ ID NO:2; and (3) a multimerization component comprising amino acids 232 to 457 of SEQ ID NO:2.

11) A method for the treatment of the manifestation of NVG including increased intraocular pressure and anterior segment neovascularization comprising administering a non-antibody VEGF antagonist to a subject in need thereof.

12) A method according to claim 11
wherein the treatment is administered to a subject who has been established to have NVI of grade 3 or 4 or/and NVA of grade 3 or 4.

13) A method according to claim 11
wherein the treatment is administered to a subject who has been established to have peripheral anterior synechiae and/or closure of the anterior chamber angle.

14) A method according to claim 11
wherein said treatment comprises sequentially administering to the subject
i.) a single initial dose of the non-antibody VEGF antagonist
ii) one or more secondary doses which are administered 5, 6, 7, 8, or 9 weeks after the immediately preceding dose to the subject who has been established to have an TOP of higher than 21 mmHg and a persistent or incomplete regression of anterior segment neovascularization at 5, 6, 7, 8, or 9 weeks after the immediately preceding dose.

15) A method according to claim 14
wherein one secondary dose is administered 5, 8, or 9 weeks after the single initial dose to the subject who has been established to have an IOP of higher than 21 mmHg and a persistent or incomplete regression of anterior segment neovascularization at 5, 8, or 9 weeks after the single initial dose.

16) A method according to claim 11
wherein said treatment is combined with IOP lowering therapy.

17) A method according to claim 16
wherein said IOP lowering therapy is selected from the group of
Carbonic anhydrase inhibitors
Intravenous hyperosmotic agents
Prostaglandin (PG) analog
Sympatholytic agent
Carbonic anhydrase inhibitor (CAI)
Sympathomimetic agent
Rho-kinase inhibitor
Laser Panretinal Photocoagulation
Laser Iridotomy
Laser Trabeculoplasty
Surgical procedures aimed at controlling increased intraocular pressure, such as trabeculectomy or implantation of devices such as valves or shunts.

18) A method according to claim 11
wherein said non-antibody VEGF antagonist comprises a VEGF fusion protein or preferably aflibercept.

19) A method according to claim 11
wherein said non-antibody VEGF antagonist comprises a VEGF fusion protein encoded by the nucleic acid sequence of SEQ ID NO: 1.

20) A method according to claim 11
wherein said non-antibody VEGF antagonist comprises a VEGF fusion protein comprising (1) a VEGFR1 component comprising amino acids 27 to 129 of SEQ ID NO:2; (2) a VEGFR2 component comprising amino acids 130 to 231 of SEQ ID NO:2; and (3) a multimerization component comprising amino acids 232 to 457 of SEQ ID NO:2.
